(19) United States
(12) Patent Application Publication
(10) Pub. No.: US 2002/0161365 A1
(43) Pub. Date: Oct. 31, 2002

(54) Method of incision/excision/coagulation of scleral, peri-scleral, iris and corneal tissues with a bipolar pulsed high frequency diathermy tip

(76) Inventor: Heraldo Martins, Recife (BR)

Correspondence Address: HERALDO MARTINS, 23 PARK STREET 01754, MAYNARD, MA, 01754, US

(21) Appl. No.: 09/842,515

(22) Filed: Apr. 25, 2001

(51) Int. Cl. A61B 18/18

(52) U.S. Cl. 606/50

(57) ABSTRACT

This invention relates to the use of a high frequency bipolar diathermy tip to perform incisions, volatilization and coagulation of tissues necessary in the surgeries of presbyopia, glaucoma and cataract. This tip acts on the sclera, cornea, iris, trabecular meshwork and peri-scleral tissues, such as Tenon's capsule, performing incisions, volatilization(excision) and/or coagulation on a very controlled way, replacing what was thought to be possible only with very expensive lasers. The method allows for very little damage on surrounding tissues. The diathermy tip delivers plasma, in a pulsed way, for a safe and effective treatment.

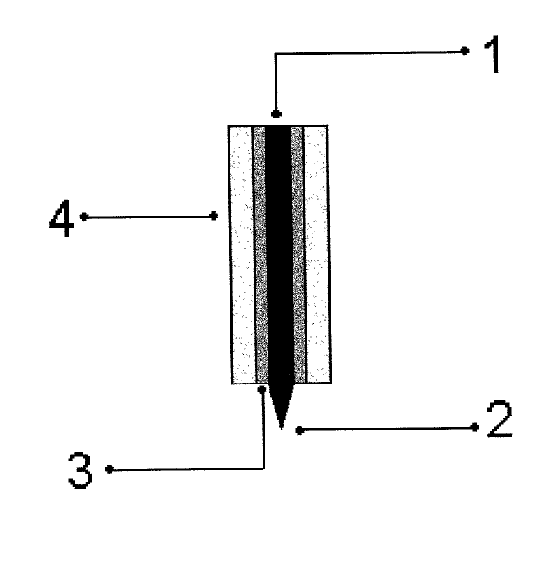

### BACKGROUND OF THE INVENTION

1. Field of Invention

The invention relates to a way of incising ocular tissues that bleed in such surgeries as presbyopia, trabeculectomy(glaucoma), and cataract. Our novel method and device allows not only to perform the incision on a very controlled way on what refers to length and depth but also remove tissue and control bleeding with minimal damage of surrounding tissues provoked by energy spillover.

2. Description of Prior Art

Incisions for surgeries of cataract, glaucoma and presbyopia have been done by means of blades or lasers. The blades are usually made out of metal or gems such as diamonds or sapphires. The lasers are usually photoablatives (excimer) photovaporizatives(CO2) and infrared(erbium). The later ablates and coagulates. The first obvious common limitation of these methods is high cost. Furthermore, blades do not coagulate nor volatize tissues.

In U.S. Pat. No. 5,346,491 it is described an apparatus for eletrical capsulotomy. Our invention relates to the use of the feed device described by Oertli in a novel way, with some other tip alterations for other completely different applications in ophthalmology.

The object of our invention is to give the surgeon na instrument and method of precisely place incisions with control of lenght, depth and bleeding. Furthermore, provide for tissue volatilization where it is desired. The physician can now perform presbyopia, glaucoma and cataract surgery in a more straightfoward way.

### SUMMARY OF THE INVENTION

The invention consists of a novel use of pre-existing commercial radio frequency unit to perform controlled incisions, volatilization and/or coagulation of tissues during presbyopia, glaucoma and cataract surgery. The pre-existing commercial models are intended to perform electrical capsulotomy, immersed in ionizing solution. In our case, the pulsed diathermy tip actuates in or out of an ionizing solution. When incising/excising the sclera it acts directly upon the tissue or, at the most, slightly wet. When incising/excising the iris it is immersed in the aqueous humor, that fills the anterior chamber of the eye. The insulated tip central wire must conform to the application. It is preferably conical in shape and with a diameter that varies with the application, usually between 50 and 200 microns and a lenght that varies usually between 50 to 600 microns.

The high-frequency current that reaches the bipolar tip actuates due to the phenomenon of dielectric hysteresis, where cutting, heating and volatilization are produced. By using this method, low power is used, avoiding burning and charring, not extending damage to surrounding tissues.

### OBJECTIVES AND ADVANTAGES OF THE INVENTION

We will describe these goals according to the type of surgical intervention

### a-Presbyopia

In this intervention the scleral is incised in a depth that goes as deep as to reach the uvea. Incisions start at the limbus and extend posteriorly for three to five milimiters. Blade incisions do not work in this surgery for it is desirable not only to incise but also remove tissue, leaving a gap incision. Infrared lasers do this task, at a high cost. Our tip does the same task incising and removing tissue, leaving a gap incision, faster and easily. Fiber optic lasers and scanning lasers need a time consuming calibration process. Our radio-frequency incision allows to obtain the same result, with the same precision.

### b-Glaucoma

In the surgical procedure known as trabeculectomy a partial thickness flap of sclera has to be construed, extending anteriorly from the sclera to the cornea, for about three milimiters. In order to do so, a preset diamond blade is used for delimitation of this flap and then a bipolar cautery forcep is used to coagulate the bleeding capilaries. But with our tip the partial depth flap cand be delimited and coagulated with one single pass. Further, a piece of the junction of the cornea and sclera (trabeculum) has to be removed by means of trephines or blades or both, with the risk of bleeding. With our tip this tissue is simply incised, vaporised, and cauterized at once, making the procedure simpler, safer and more effective. More ahead it is needed to remove a small piece of the base of the iris (iridectomy). This stage of the surgical procedure is accomplished usually my meas of pinching the iris with a forceps anc cutting it with scissors. With our tip we just need to lightly scrug the base of the iris and it is done, without bleeding. Billions of dollars are spent every year with glaucoma eye drops and thousands of persons go blind. With this novel tool surgical procedure becomes easier and safer to perform.

### c-Cataract

This is the most performed surgical procedure in the world today. In order to enter the anterior chamber and perform the surgery, two methods are employed. One direct method, which is called clear cornea and a scleral approach, where a tunnel from the sclera to the cornea is construed. In this latter approach the entrance of the tunel is a partial depth incision made on the sclera with a preset depth. This incision is made and then cauterized. With our tip the incision is precisely placed and no need cauterization or expensive diamond blades or disposable blades.

### DETAILED DESCRIPTION OF THE PREFERRED EMBODIMENTS

Our method of incising sclera, cornea and iris is different from all prior methods employed in the surgical procedures for presbyopia, glaucoma and cataract. With this high frequency, pulsed, insulated, self-limited bipolar diathermy tip, the physician can achieve controlled depth, controlled coagulation and incision lenth with a single instrument.

Using commercially available radiofrequency units designed for electrical capsulotomy various tip configurations can be designed, according to surgeon preference and type of surgery. With the commercially available radio-frequency generators, frequencies of 500 Kz are tipically used. Pulse duration and pause are emplyed to minimize thermal damage and to use less energy. But some thermal efffect is desirable to achieve mild coagulation, necessary when incising vascularized tissue.

The preferred configuration of the tip is that of a straight metallic wire, varying in diameter from 50 to 300 microns, in a tight conduit, coated by an external metallic cilindrical shield shield. The inner wire is the active pole and protrudes preferably on a conical shape from 100 to 600 microns from where the conduit and outer coating stops. Being a bipolar tip, the outer coating is the passive pole. Dielectric effect is created from the flow of current from the inner wire to the outer coating, disrupting atoms and generating a very contained heat.

In some instances the surgeon may prefer the distal portion of the tp to be bent 90 degrees or maybe to be curvelinear. It is a matter of personal choice and the type of surgery it is designed for. The hi-frequency pulsed tip does not need any special gas or fluid to act. It simply touches the tissue it needs to act upon, be it the surface of the sclera or inside the anterior chamber, incising the iris. A commom lenght for manufacturing the tip from the active wire tip to the distal end where it connects to the bipolar cable is 2.0 cm. But it is a preference of the manufacturer and does not interfere on the method. Tips with shorter exposed active wire, e.g 100 microns, will make shallower incisions on the sclera. Incision depth is also dependent, to some extent, on the pressure exerted on the tip by the surgeon, by tissue hidration and by current parameters .

The electro incision unit made with these characteristics for the novel uses herein explained are efficient due to the pulsed mode and work on low power when compared to classical units. This is a good reason why little or negligible damage occurs to surrounding tissue, which is highly desirable when acting upon the eye.

I claim:

1. A high frequency bipolar pulsed diathermy tip for aplication in the surgical correction of presbyopia by means of incision, coagulation and volatilization of scleral tissue
said diathermy tip being connected to radio frequency units primarily used to perform electrical capsulotomy;
said diathermy tip intended to perform controlled incision on the sciera with or without ionic solutions
said diatermy tip being straight, bent or curvelinear; said diatermy tip comprising an inner insulated active wire and an outer passive coating;
said innerwire extending from 100 to 600 microns outward from the border of the insulation and the passive coating;
said inner wire having preferably but not restricted to a conical shape;
said inner wire having a diameter in between 50 and 300 microns at the base of the cone;

2. A high frequency bipolar pulsed diathermy tip for aplication in the surgical correction of glaucoma by means of incision, coagulation and volatilization of scleral tissue, coneal tissue and iris tissue;
said diathermy tip being connected to radio frequency units primarily used to perform electrical capsulotomy;
said diathermy tip performing in the presence or abscence of ionizing solutions;
said diatermy tip being straight, bent or curvelinear;
said diatermy tip comprising an inner insulated active wire and an outer passive coating;
said inner wire extending from 100 to 600 microns outward from the border of the insulation and the passive coating;
said inner wire having preferably but not restricted to a conical shape;
said inner wire having a diameter in between 50 and 300 microns at the base of the cone;

3. A high frequency bipolar pulsed diathermy tip for aplication in the surgical correction of cataract, performing the preset depth incision;
said diathermy tip being connected to radio frequency units primarily used to perform electrical capsulotomy;
said diathermy tip performing in the presence or abscence of ionizing solutions;
said diatermy tip being straight, bent or curvelinear;
said diatermy tip comprising an inner insulated active wire and an outer passive coating;
said inner wire extending from 100 to 600 microns outward from the border of the insulation and the passive coating;
said inner wire having preferably but not restricted to a conical shape;
said inner wire having a diameter in between 50 and 300 microns at the base of the cone;
